(19) United States
(12) Patent Application Publication
(10) Pub. No.: US 2008/0058287 A1
(43) Pub. Date: Mar. 6, 2008

(54) Mesotherapy Cream

(75) Inventors: John Rose, Phoenix, AZ (US); Nyangenya Maniga, Gilbert, AZ (US)

Correspondence Address: JENNINGS, STROUSS & SALMON, P.L.C., 201 E. WASHINGTON ST., 11TH FLOOR, PHOENIX, AZ, 85004, US

(73) Assignee: Natural Designs

(21) Appl. No.: 11/849,046

(22) Filed: Aug. 31, 2007

Related U.S. Application Data
(60) Provisional application No. 60/842,198, filed on Aug. 31, 2006.

(51) Int. Cl. A61K 31/56; A61K 31/66; A61P 43/00

(52) U.S. Cl. 514/109; 514/171

(57) ABSTRACT

The present disclosure concerns a cream-based composition and method which reduces fat deposits upon topical application to the skin. The active ingredients of the composition include the active ingredients deoxycholic acid and phosphatidyl choline which are mixed together with a cream composition. The active ingredients may be combined with any suitable pharmaceutical vehicle to provide the fat-reducing composition claimed herein. The composition is topically applied to a skin area over a period of days or months in order to reduce fat deposits that have collected beneath the dermis.

### CLAIM TO DOMESTIC PRIORITY

This application claims the benefit of priority of provisional application Ser. No. 60/842,198 filed Aug. 31, 2006.

### FIELD OF THE INVENTION

This invention relates generally to the field of compositions and methods for regulating body weight, especially for promoting body weight loss, for maintaining or controlling body weight, reducing levels of fat deposits and facilitating or promoting metabolism of fat deposits, and more specifically to a composition and method for a cream that promotes the leaching of fatty deposits into the blood stream, where it can be more easily metabolized and used for energy.

### BACKGROUND OF THE INVENTION

About 97 million adults in the United States are overweight or obese. The medical problems caused by overweight and obesity can be serious and often life-threatening, and include diabetes, shortness of breath, gallbladder disease, hypertension, elevated blood cholesterol levels, cancer, arthritis, other orthopedic problems, reflux esophagitis (heartburn), snoring, sleep apnea, menstrual irregularities, infertility and heart trouble. Moreover, obesity and overweight substantially increase the risk of morbidity from hypertension, dyslipidemia, type 2 diabetes, coronary heart disease, stroke, gallbladder disease, osteoarthritis and endometrial, breast, prostate, and colon cancers.

Higher body weights are also associated with increases in all-cause mortality. Most or all of these problems are relieved or improved by permanent significant weight loss. Longevity is likewise significantly increased by permanent significant weight loss.

### DETAILED DESCRIPTION OF VARIOUS EMBODIMENT

The present disclosure provides for a novel composition and method for reducing fat deposits in the human body. More specifically, the present disclosure concerns a cream-based composition and method which reduces fat or fat deposits upon topical application to the skin, also referred to here as mesotherapy cream. The active ingredients of the composition include deoxycholic acid and phosphatidyl choline, which are mixed together with a cream composition. The active ingredients may be combined with any suitable pharmaceutical vehicle to provide the fat-reducing composition claimed herein.

The composition is topically applied to a skin area over a period of days or months in order to reduce fat deposits that have collected beneath the dermis. More specifically, the cream-based composition is applied to the human skin and, when absorbed into the dermis, promotes the breakdown and leaching of fat deposits beneath the dermis. The leaching of the fat deposits releases the fat into the blood stream where the fat can more easily be metabolized and can be used for energy or excreted by normal processes in the liver.

The novelty of this disclosure rests in the combination of active ingredients used herein combined with a cream base or other pharmaceutically excepted carrier that can facilitate the absorption of the active ingredients into and through the dermis where they can act on fat deposits residing thereunder.

According to the present disclosure the active ingredients are present in the cream as follows: deoxycholic acid, preferably in an amount ranging from 0.5% to 10% of the composition and phosphatidyl choline, preferably in an amount ranging from 0.1% to 5% of the composition. In one embodiment of the disclosure, Phospholipon 90G is used as the source of the phosphatidyl choline component.

The following Table 1 illustrates alternate embodiments of the disclosed composition. By way of example only, Table 1 provides the disclosed ranges for each ingredient in one embodiment of the composition. The ranges are given in a percent of the entire composition for each ingredient. It is recognized, however, that any cream base or other acceptable topical pharmaceutical carrier may be used as the carrier for the active ingredients disclosed herein.

TABLE 1

| Ingredient | Range % of composition |
|---|---|
| Water | 100-total of other ingredients |
| Glycerin | 1-10 |
| Propylene glycol | 0.0-30 |
| Sodium PCA | 0.5-10 |
| Methyl paraben | 0.05-1 |
| Potassium sorbate | 0.05-0.5 |
| Disodium EDTA | 0.0-1 |
| Diazolidinyl Urea | 0.0-0.2 |
| Allantoin | 0.1-1 |
| Ascorbyl palmitate | 0.05-1 |
| Triethanolamine | 0.05-0.8 |
| Deoxycholic Acid | 0.5-10 |
| Phosphatidyl choline | 0.5-5 |
| Sodium hyaluronate | 0.01-1 |
| Carbomer | 0.05-1 |
| Glyceryl stearate SE | 0.5-3 |
| Capric/capyrylic Triglycerides | 0.5-20 |
| Stearic acid | 0.5-5 |
| Cetearyl Alcohol and cetearate-20 | 0.0-1 |
| Isopropyl palmitate | 1-10 |
| Propyl; paraben | 0.05-1 |
| Sorbic acid | 0.05-0.2 |
| Shea Butter | 0.5-20 |
| Retinyl palmitate | 0.05-1 |
| Tocopheryl acetate | 0.05-1 |

The following Table 2 illustrates one specific embodiment of the disclosed composition.

TABLE 2

| Ingredient | Amount as a % of the composition |
|---|---|
| Part A | |
| Water | 59.25 |
| Glycerin | 5 |
| Sodium PCA | 0.5 |
| Methyl paraben | 0.2 |
| Disodium EDTA | 0.1 |
| Diazolidinyl Urea | 0.1 |
| Allantoin | 0.7 |
| Ascorbyl palmitate | 0.05 |
| Triethanolamine | 0.8 |
| Deoxycholic Acid | 5 |
| Phosphatidyl choline | 1.5 |
| Sodium hyaluronate 1% | 1 |
| Part B | |
| Carbomer | 0.5 |
| Water | 15 |
| Part C | |
| Glyceryl stearate SE | 1 |
| Capric/capyrylic Triglycerides | 1 |
| Stearic acid | 1 |
| Cetearyl Alcohol and cetearate-20 | 1 |
| Isopropyl palmitate | 4 |
| Propyl; paraben | 0.1 |
| Shea Butter | 2 |
| Retinyl palmitate | 0.1 |
| Tocopheryl acetate | 0.1 |
| Total | 100 |

Generally, in one embodiment, the ingredients are combined as follows. Weigh the ingredients in Part A and let soak for a day. Weight the ingredients in Part B after Part A has soaked. Heat the ingredients in Part A and in Part B separately until each mixture reaches a temperature between 185-195° F. Add Part B to Part A while mixing and homogenizing. Weigh and heat Part C to 185° F. Add Part C to the already combined Parts A+B. Homogenize the entire mixture until it reaches a smooth cream consistency.

Table 3 illustrates another specific embodiment of the disclosed composition as it would be prepared in an 800 lb batch.

TABLE 3

| Ingredient | Range % | Batch weight in lbs. |
|---|---|---|
| A | | |
| Water | 59.25 | 474 |
| Glycerin | 5 | 40 |
| Sodium PCA | 0.5 | 4 |
| Methyl paraben | 0.2 | 1.6 |
| Disodium EDTA | 0.1 | 0.8 |
| Diazolidinyl Urea | 0.1 | 0.8 |
| Allantoin | 0.7 | 5.6 |
| Ascorbyl palmitate | 0.05 | 0.4 |
| Triethanolamine | 0.8 | 6.4 |
| Deoxycholic Acid | 5 | 40 |
| Phosphatidyl choline | 1.5 | 12 |
| Sodium hyaluronate 1% | 1 | 8 |
| B | 0 | 0 |
| Carbomer | 0.5 | 4 |
| Water | 15 | 120 |
| C | 0 | 0 |
| Glyceryl stearate SE | 1 | 8 |
| Capric/capyrylic Triglycerides | 1 | 8 |
| Stearic acid | 1 | 8 |
| Cetearyl Alcohol and cetearate-20 | 1 | 8 |
| Isopropyl palmitate | 4 | 32 |
| Propyl; paraben | 0.1 | 0.8 |
| Shea Butter | 2 | 16 |
| Retinyl palmitate | 0.1 | 0.8 |
| Tocopheryl acetate | 0.1 | 0.8 |
| | 0 | 0 |
| Total | 100 | 800 |

The following illustrates one specific embodiment of the manufacturing process for the disclosed composition using the 800 lb batch described in Table 3. However, it is recognized that the particular embodiment disclosed herein may be scaled up or down depending on the desired quantity of the composition to be made.

First, weigh 474 pounds (lb) water into a 200 gallon stainless double jacket tank (Lee industries model). The tank is equipped with a dual sweep mixer rotating in opposite directions for proper mixing. Weigh and add into tank the following: 40 lb glycerin, 4 lb sodium PCA, 1.6 lb methyl paraben, 0.8 lb disodium EDTA, 0.8 lb diazolidinyl Urea, 5.6 lb allantoin, 0.8 lb ascorbyl palmitate, and 6.4 lb triethanolamine. Mix until all solids have dissolved. Stop the mixer.

Then add 40 lb deoxycholic acid, 12 lb phosphatidyl choline and 8 lb sodium hyaluronate 1%. Start the mixer and mix at moderate speed. Turn on the steam heating system and heat until the temperature is about 185° F. This is referred to herein as the “Part A” mixture and will be used as described below.

Next, weigh 120 lb water into a 40 gallon stainless steel tank (Lee industries). Add 4 lb carbopol ultrez 10 slowly on the surface. Do not mix or agitate. Let the carbopol ultrez 10 powder hydrate and dissolve in water. When dissolution is complete, mix until a uniform solution has formed. This solution is referred to as the carbopol or “Part B” solution and will be used as described below.

When the temperature of Part A is about 180° F. add slowly, mixing the carbopol solution from Part B above and heat until the temperature is about 180° F. This is now referred to as Part A+B and used as described below.

Next, weigh the following ingredients and place in a 60 gallon double jacketed stainless steel tank as follows. Weigh 8 lb each of glyceryl stearate SE, capric/capyrylic triglycerides, stearic acid, cetearyl Alcohol and cetearate-20 and place in tank. Also weigh and place into tank each of the following 32 lb isopropyl palmitate, 0.8 lb propyl paraben, 16 lb Shea butter, 0.8 lb retinyl palmitate, and 0.8 lb tocopheryl acetate. Heat the mixture while mixing until 185° F. This is referred to as the “Part C” mixture.

Finally, when the temperature of part C is 185° F. and that of the mixture of parts A+B is 185° F., stop heating and add slowly part C into mixture A+B while mixing at high speed. At the completion of addition of part C, continue mixing for another 15 minutes. A cream is formed after blending Parts C and A+B. Let the cream cool down to 140° F. Homogenize the cream using a Silverson mill operating at 60 rpm for 30 minutes. A fine cream is obtained.

This cream is absorbed quickly when spread over the surface of skin. The cream or other topical preparation including the active ingredients above acts directly on the fat deposits beneath the dermis. The cream does not require metabolism by the liver or kidneys and therefore is advantageous over existing oral medication for obesity or weight loss.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of a preferred embodiment and best mode of the invention known to the applicant at this time of filing the application has been presented and is intended for the purposes of illustration and description. It is not intended to be exhaustive nor limit the invention to the precise form disclosed and many modifications and variations are possible in the light of the above teachings. The embodiment was chosen and described in order to best explain the principles of the invention and its practical application and to enable others skilled in the art to best utilize the invention in various embodiments and with various modifications as are suited to the particular use contemplated. Therefore, it is intended that the invention not be limited to the particular embodiments disclosed for carrying out the invention.

What is claimed is:

1. A composition for reducing fat deposits in the human body comprising: deoxycholic acid and phosphatidyl choline.

2. The composition of claim 1, wherein the deoxycholic acid is present in amount in a range of approximately 0.5 to 10 % by weight.

3. The composition of claim 1, wherein the phosphatidyl choline is present in a range of approximately 0.1 to 5% by weight.

4. The composition of claim 1, further including a pharmaceutically acceptable carrier.

5. The composition of claim 4, wherein the pharmaceutically acceptable carrier facilitates absorption of the composition into the dermal layer of a human.

6. The composition of claim 1, further including a cosmetic cream.

7. The composition of claim 1, wherein the phosphatidyl choline is Phospholipon 90G.

8. The composition of claim 1, wherein the composition reduces fat deposits in the human by leaching fat cells into a human's blood stream.

9. A method for reducing fat deposits in the human body comprising: administering a composition comprising deoxycholic acid and phosphatidyl choline.

10. The method of claim 8, wherein the composition further includes a cosmetic cream.

11. The method of claim 8, wherein the composition is administered by applying the composition to the skin of a human.

12. The method of claim 10, wherein the composition is absorbed into the skin of a human and contacts fat deposits beneath the human's skin.

13. The method of claim 11, wherein the composition breaks down fat deposits by leaching fat cells into a human's blood stream.

14. The method of claim 11, wherein fat cells are emulsified and dislodged from localized fat deposits.

15. The method of claim 12, wherein the dislodged fat cells flow into the blood stream, wherein the fat cells are metabolized.

16. A method for manufacturing a fat-reducing composition, comprising: combining deoxycholic acid and phosphytidyl choline with a pharmaceutically acceptable carrier.

17. The method of claim 15, wherein the deoxycholic acid is combined in amount in a range of approximately 0.5 to 10 % by weight.

18. The method of claim 15, wherein the phosphatidyl choline is combined in a range of approximately 0.1 to 5% by weight.

19. The method of claim 18, wherein the pharmaceutically acceptable carrier is a cosmetic cream.

20. The method of claim 19, wherein the cosmetic cream facilitates absorption of the composition into the human skin allowing the composition to contact fat deposits beneath the human's skin.
